(19) United States
(12) Patent Application Publication
(10) Pub. No.: US 2023/0329997 A1
(43) Pub. Date: Oct. 19, 2023

(54) AN ANTIPERSPIRANT COMPOSITION

(71) Applicant: Conopco, Inc., d/b/a UNILEVER, Englewood Cliffs, NJ (US)

(72) Inventors: Xiaoke LI, Shanghai (CN); Weichong WANG, Shanghai (CN)

(73) Assignee: Conopco, Inc., d/b/a UNILEVER, Englewood Cliffs, NJ (US)

(21) Appl. No.: 18/026,847

(22) PCT Filed: Sep. 17, 2021
(86) PCT No.: PCT/EP2021/075553

§ 371 Date: Mar. 17, 2023

(30) Foreign Application Priority Data
Oct. 14, 2020 (WO) PCT/CN2020/120788
Nov. 16, 2020 (EP) 20207869.7

(51) Int. Cl. A61K 8/73

(52) U.S. Cl. CPC A61K 8/731; A61K 2800/874; A61K 2800/77; A61K 2800/413

(57) ABSTRACT

The present invention is in the field of antiperspirant compositions, in particular, compositions comprising antiperspirant actives. Disclosed is an aqueous antiperspirant composition comprising: (i) nanocrystalline cellulose with a length from 1 run to 300 nm and a diameter from 1 nm to 20 nm; and (ii) a cosmetically acceptable carrier, wherein the ionic strength of the composition is lower than 0.016; wherein said composition is not a pickering emulsion; wherein surface of said nanocrystalline cellulose comprises hydroxyl and sulfate groups, wherein said composition comprises from 1 to 20 wt % of said nanocrystalline cellulose based on total weight of the composition.

### FIELD OF THE INVENTION

The present invention is in the field of antiperspirant compositions.

### BACKGROUND OF THE INVENTION

The present invention relates to compositions, such as those that contain antiperspirant actives. These actives are added to compositions to reduce perspiration upon topical application to the body, particularly to the underarm regions of the human body viz. the axilla and sometimes even on the upper part of the body near the chest.

Usually, conventional antiperspirant actives are salts of metals having an astringent effect, such as the salts of aluminum and/or zirconium. Since antiperspirants are used regularly, and have been used for decades, there is an ever increasing need to develop alternative antiperspirant actives which are equally efficacious.

WO2013033833 A1 (BIOVISION TECHNOLOGY INC.) discloses a topical composition for application to the skin of a mammal, and the topical composition comprising nanocrystalline cellulose. The nanocrystalline cellulose is manufactured according to WO2011072365 A1 which provides a process for producing cellulose nanocrystal with carboxylic acid on the surface.

US20180015194 A1 (ZEON CORPORATION) discloses a deodorant comprising metal-containing oxidized cellulose nanofibers. The cellulose are highly crystalline ultrafine fibers and are carboxylated cellulose nanofibers.

US20140037816 A1 (HERCULES INCORPORATED) discloses a stabilized multiphase composition comprising a first phase, a second phase, cellulose ether and nanocrystalline cellulose. It discloses that nanocrystalline cellulose in combination with water soluble cellulose ethers can act as stabilizer for multiple systems.

US20190224083 A1 (NIPPON PAPER INDUSTRIES CO., LTD.) discloses a foamable aerosol composition comprising a stock solution comprising a cellulose nanofiber and a propellant.

### SUMMARY OF THE INVENTION

The present inventors have surprisingly found that certain amount of nanocrystalline cellulose (NCC) with a length from 1 nm to 300 nm and a diameter from 1 nm to 20 nm, which has a high degree of crystallinity in nature and surface of said nanocrystalline cellulose comprises hydroxyl and sulfate groups, are stable in aqueous composition with low ionic strength. In addition to being stable, the particle is capable of forming aggregation when they contact with aqueous saline media, which is equivalent to perspiration or sweat, thereby is capable of preventing sweat from rapidly being released. In this manner, such nanocrystalline cellulose (NCC) behave much like conventional antiperspirants. The nanocrystalline cellulose (NCC) of the present invention has a length from 1 nm to 300 nm and a diameter from 1 nm to 20 nm, and high degree of crystallinity which distinguishes them from other cellulose nanofibrils and microcrystalline cellulose.

In accordance with a first aspect is disclosed an aqueous antiperspirant composition comprising:
(i) nanocrystalline cellulose with a length from 1 nm to 300 nm and a diameter from 1 nm to 20 nm; and
(ii) a cosmetically acceptable carrier,

wherein the ionic strength of the composition is lower than 0.016; wherein said composition is not a Pickering emulsion; wherein surface of said nanocrystalline cellulose comprises hydroxyl and sulfate groups, wherein said composition comprises from 1 to 20 wt % of said nanocrystalline cellulose based on total weight of the composition.

In accordance with a second aspect is disclosed a method of reducing perspiration comprising a step of topical application of the composition of the first aspect.

In accordance with a third aspect is disclosed use of the composition of the first aspect for reduction of bodily perspiration.

All other aspects of the present invention will more readily become apparent upon considering the detailed description and examples which follow.

Except in the examples, or where otherwise explicitly indicated, all numbers in this description indicating amounts of material or conditions of reaction, physical properties of materials and/or use may optionally be understood as modified by the word “about”.

All amounts are by weight of the antiperspirant composition, unless otherwise specified.

It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

### DETAILED DESCRIPTION OF THE INVENTION

By “An antiperspirant Composition” as used herein, is meant to include a composition for topical application to the skin of mammals, especially humans. Such a composition is preferably of the leave-on type. By a leave-on composition is meant a composition that is applied to the desired skin surface and left on for a minute to 24 hours after which it may be wiped or rinsed off with water, usually during the regular course of personal washing. The composition may also be formulated into a product which is applied to a human body for improving the appearance, cleansing, odor control or general aesthetics.

The composition of the present invention can be in the form of a liquid, lotion, cream, foam, scrub, gel or stick form and may be delivered through a roll-on device or using a propellant containing aerosol can. It is especially useful for delivering low pH compositions to the axilla of an individual for antiperspirant benefits. “Skin” as used herein is meant to include skin on any part of the body (e.g., neck, chest, back, arms, underarms, hands, legs, buttocks and scalp) especially the underarms.

By “Pickering emulsion” as used herein, is meant to be an emulsion that is stabilized by solid particles (for example colloidal silica) which adsorb onto the interface between the two phases. Preferably, the Pickering emulsion used herein is the emulsion stabilized by nanocrystalline cellulose particles.

By “water-soluble” as used herein, is meant to be a salt that has a solubility in water at 20° C. and 1 atm of greater than 1 g salt/100 ml water.

Nanocrystalline Cellulose (NCC)

Nanocrystalline cellulose (NCC), also known as cellulose nanocrystals (CNC), is cellulose in crystalline form, and it is rod-like particle sourced from natural materials. It is usually generated by the removal of amorphous sections of a purified cellulose source by acid hydrolysis.

Antiperspirant compositions in accordance with this invention comprise nanocrystalline cellulose with a length from 1 nm to 300 nm and a diameter from 1 nm to 20 nm. Preferably, the nanocrystalline cellulose has a length from 10 nm to 200 nm and a diameter from 1.5 to 10 nm. More preferably, the nanocrystalline cellulose has a length from 30 nm to 150 nm and a diameter from 2 nm to 5 nm.

Such kind of nanocrystalline cellulose is usually in form of powder and can form stable dispersion in aqueous solution.

Antiperspirant composition in accordance with this invention comprises nanocrystalline cellulose that the surface of nanocrystalline cellulose preferably comprises hydroxyl and sulfate groups.

Antiperspirant composition in accordance with this invention comprises from 1 to 20 wt %, preferably from 2 to 20 wt %, and more preferably from 2 to 10 wt %, and most preferably from 2 to 5 wt % nanocrystalline cellulose, based on total weight of the composition.

Without wishing to be bound by theory the inventors believe that once the nanocrystalline cellulose of the present invention is applied, when it contacts with perspiration or sweat, can form an aggregation and which thereby provides antiperspirant benefits.

Antiperspirant compositions in accordance with this invention may advantageously comprise an additional antiperspirant active. However, it is preferred that the antiperspirant composition in accordance with this invention is free from a conventional antiperspirant salt, for example, salts comprising Al and/or other forms of Zr, and/or other forms of Zn, such as aluminum chlorohydrate or aluminum-zirconium chlorohydrate optionally complexed with glycine.

More preferably, the composition of the present invention is free from metal based antiperspirant active. The metal includes aluminum, zirconium, zinc, titanium, copper, gallium, stannum, Indium, hafnium, vanadium, cobalt.

Ionic Strength

The ionic strength of a solution is a measure of the concentration of ions in that solution. Ionic compound is a compound formed by ions bonding together through electrostatic forces. Ionic compounds, when dissolved in water, dissociate into ions. One of the main characteristics of a solution with dissolved ions is the ionic strength.

It is essential to have the composition formulated with ionic strength lower than 0.016 molar (mol/L), preferably not greater than 0.012 molar, more preferably with ionic strength of 0. The composition in accordance with this invention comprises lower than 0.016 molar water-soluble ionic compound.

Without wishing to be bound by theory it is believed that at low ionic strength, the composition of the invention is more effective.

When the composition comprises a water-soluble ionic compound which is a water-soluble metal salt, it is preferred that the composition comprises lower than 0.016 molar such metal salt. Such metal salt can be but not limit to water-soluble salt of sodium, potassium, calcium, magnesium, zinc.

More preferably, the composition in accordance with this invention is free of water-soluble metal salt.

More preferably, the composition in accordance with this invention is free of water-soluble ionic compound.

Without wishing to be bound by theory the inventors believe it is essential to have the composition formulated and delivered at a lower ionic strength lower than 0.016 molar in order for the formulation to be effective.

Other Ingredients

Other components commonly included in conventional antiperspirant compositions may also be incorporated in the compositions of the present invention. Such components include skin care agents such emollients, humectants and skin barrier promoters; skin appearance modifiers such as skin lightening agents and skin smoothing agents; anti-microbial agents, in particular organic anti-microbial agents, and preservatives.

The antiperspirant compositions of the invention could be applied cosmetically and topically to the skin, broadly speaking, by one of two methods. Different consumers prefer one method or the other. In one method, sometimes called a contact method, the composition is wiped across the surface of the skin, depositing a fraction of the composition as it passes. In the second method, sometimes called the non-contact method, the composition is sprayed from a dispenser held proximate to the skin, often in an area of about 10 to 20 cm². The spray can be developed by mechanical means of generating pressure on the contents of a dispenser, such as a pump or a squeezable sidewall or by internally generated pressure arising from a fraction of a liquefied propellant volatilizing, the dispenser commonly being called an aerosol.

There are broadly speaking two classes of contact compositions, one of which is liquid and usually applied using a roll-on dispenser or possibly absorbed into or onto a wipe, and in the second of which the antiperspirant active is distributed within a carrier liquid that forms a continuous phase that has been gelled. In one variation, the carrier fluid comprises a solvent for the antiperspirant and in a second variation, the antiperspirant remains a particulate solid that is suspended in an oil, usually a blend of oils.

The composition of the invention comprises an aqueous cosmetically acceptable carrier. The term aqueous means that the composition of the invention comprises water as the main carrier or that water forms a major part of the carrier. In such cases, other solvents and ingredients other than water may also be present.

The composition of the invention is not a Pickering emulsion. It is preferred that the composition of the invention is not an emulsion, comprising one phase.

It is preferred that the composition of the invention is in the form of a cream, a spray, a firm solid, a soft solid or is an emulsion packaged in a roll-on applicator.

It is preferred that, when said composition is a spray it comprises a propellant and the composition is in the form of an aerosol.

Further preferably, the composition of the invention is in the form of a roll-on or aerosol product.

Stick Compositions

Many different materials have been proposed as gellants for a continuous oil phase, including waxes, small molecule gelling agents and polymers. They each have their advantages and of them, one of the most popular class of gellants is waxes, partly at least due to their ready availability and ease of processing, including in particular linear fatty alcohol wax gellants. A gelled antiperspirant composition is applied topically to skin by wiping it across and in contact with the skin, thereby depositing on the skin a thin film.

The nature of the film depends to a significant extent on the gellant that is employed. Although wax fatty alcohols have been employed as gellants for many years, and are effective for the purpose of gelling, the resultant product is rather ineffective at improving the visual appearance of skin, and in particular underarm skin, to which the composition has been applied. This problem has been solved by including ameliorating materials for example, di or polyhydric humectants and/or a triglyceride oil.

Stick compositions are usually available in the form of a firm solid or a soft solid. Firm solids, as the name indicates, are harder and can be directly applied by way of an applicator, for example, to the underarms. Soft solids also need an applicator which is similar to the firm solids, the difference being that the soft solids are softer and the applicator needs to be designed in order to permit extrusion of the solids through a cap member comprising plurality of orifices and the extruded composition can then be applied to the underarms.

Roll-on

Liquid compositions that are applicable from a roll-on broadly speaking can be divided into two classes, namely those in which an antiperspirant active is suspended in a hydrophobic carrier, such as a volatile silicone and those in which the antiperspirant active is dissolved in a carrier liquid. The latter has proven to be more popular. There are mainly two sorts of dissolving carrier liquid, namely carriers that are predominantly alcoholic, which is to say the greater part of the dissolving carrier fluid comprises ethanol and the second class in which the carrier liquid is mainly water. The former was very popular because ethanol is a mild bactericide in its own right, but its popularity waned because it stings, especially if the surface onto which the composition has been applied has been damaged or cut, such as can easily arise during shaving or other depilatory actions.

The second class of formulations that is an alternative to alcoholic formulations comprise a dispersion of water-insoluble or very poorly water-soluble ingredients in an aqueous solution of the antiperspirant. Herein, such compositions will be called emulsions. Antiperspirant roll-on emulsions commonly comprise one or more emulsifiers to maintain a distribution of the water-soluble ingredients.

Aerosol Compositions

The antiperspirant composition may be delivered through an aerosol composition which comprises a propellant in addition to the other ingredients described hereinabove. Commonly, the propellant is employed in a weight ratio to the base formulation of from 95:5 to 5:95. Depending on the propellant, in such aerosol compositions the ratio of propellant to base formulation is normally at least 20:80, generally at least 30:70, particularly at least 40:60, and in many formulations, the weight ratio is from 90:10 to 50:50. A ratio range of from 70:30 to 90:10 is sometimes preferred.

Propellants herein generally are one of three classes; i) low boiling point gasses liquified by compression, ii) volatile ethers and iii) compressed non-oxidising gases.

Class i) is conveniently a low boiling point material, typically boiling below −5° C., and often below −15° C., and in particular, alkanes and/or halogenated hydrocarbons. This class of propellant is usually liquefied at the pressure in the aerosol canister and evaporates to generate the pressure to expel the composition out of the canister. Examples of suitable alkanes include particularly propane, butane or isobutane. The second class of propellant comprises a very volatile ether of which the most widely employed ether hitherto is dimethyl ether. This propellant can advantageously be employed at relatively low weight ratio of propellant to base formulation, for example to as low as 5:95. It can also be employed in admixture with, for example, compressible/liquefiable alkane gasses. The third class of propellant comprises compressed non-oxidising gasses, and in particular carbon dioxide or nitrogen. Inert gases like neon are a theoretical alternative.

The composition of the invention comprises a topically acceptable carrier. When the composition of the invention is in the form of a roll-on, a cream, a spray, or a firm solid or a soft solid, the topically acceptable carrier comprises an anhydrous carrier or an aqueous carrier. The anhydrous carrier in such cases may comprise a silicone compound, low boiling alcohol or a wax. When the composition comprises a propellant, it is delivered as an aerosol.

The composition of the present invention can comprise a wide range of other optional components. The CTFA Personal Care Ingredient Handbook, Second Edition, 1992, which is incorporated by reference herein in its entirety, describes a wide variety of non-limiting personal care and pharmaceutical ingredients commonly used in the skin care industry, which are suitable for use in the compositions of the present invention. Examples include: antioxidants, binders, biological additives, buffering agents, colorants, thickeners, polymers, astringents, fragrance, conditioners, exfoliating agents, pH adjusters, other than the ones already discussed earlier, preservatives, natural extracts, essential oils, skin sensates, skin soothing agents, and skin healing agents.

A preservative is a preferred additional component in compositions of the invention. A preservative serves to reduce or eliminate microbial contamination of compositions of the invention. Preservatives are typically employed at a total level of from 0.05 to 3%, preferably at from 0.1 to 2% and most preferably at from 0.4 to 1%.

Suitable preservatives for use with the present invention include 2-phenoxyethanol, iodopropynyl butylcarbamate, C1-C3 alkyl parabens, sodium benzoate, caprylyl glycol and EDTA. Particularly preferred preservatives are 2-phenoxyethanol, iodopropynyl butylcarbamate, sodium benzoate, caprylyl glycol and EDTA and especially preferred are 2-phenoxyethanol and iodopropynyl butylcarbamate.

A preferred additional component of compositions of the invention is a fragrance. Suitable materials include conventional perfumes, such as perfume oils and also include so-called deo-perfumes, as described in EP 545,556 and other publications. Levels of incorporation are preferably up to 4% by weight, particularly from 0.1% to 2% by weight, and especially from 0.7% to 1.7% by weight.

An antimicrobial deodorant active is a preferred an additional component in compositions of the invention. Such components serve to reduce or eliminate body odour by reducing or otherwise impeding the function of microbes on the skin of the body responsible for malodour generation.

The antimicrobial deodorant active may also be a preservative for the composition.

When employed, the anti-microbial deodorant agent is typically incorporated into the composition at from 0.01% to 3% and particularly at from 0.03% to 0.5%.

Preferred anti-microbial deodorant agents have a minimum inhibitory concentration (MIC) of 1 mg.ml⁻¹ or less, particularly 200 μg.ml⁻¹ or less, and especially 100 μg.ml⁻¹ or less. The MIC of an anti-microbial agent is the minimum concentration of the agent required to significantly inhibit microbial growth. Inhibition is considered “significant” if an 80% or greater reduction in the growth of an inoculum of Staphylococcus epidermidis is observed, relative to a control medium without an anti-microbial agent, over a period of 16 to 24 hours at 37° C. Details of suitable methods for determining MICs can be found in “Antimicrobial Agents and Susceptibility Testing”, C.Thornsberry, (in “Manual of Clinical Microbiology”, 5th Edition, Ed. A. Balows et al, American Society for Microbiology, Washington D.C., 1991). A particularly suitable method is the Macrobroth Dilution Method as described in Chapter 110 of above publication (pp. 1101-1111) by D. F. Sahm and J. A. Washington II. MICs of anti-microbials suitable for inclusion in the compositions of the invention are triclosan: 0.01-10 μg.ml⁻¹ (J. Regos et al., Dermatologica (1979), 158: 72-79) and farnesol: ca. 25 μg.ml⁻¹ (K. Sawano, T. Sato, and R. Hattori, Proceedings of the 17th IFSCC International Conference, Yokahama (1992) p.210-232). By contrast ethanol and similar alkanols have MICs of greater than 1 mg.ml⁻¹.

Suitable organic anti-microbials are bactericides, for example quaternary ammonium compounds, like cetyltrimethylammonium salts; chlorhexidine and salts thereof; and diglycerol monocaprate, diglycerol monolaurate, glycerol monolaurate, and similar materials, as described in “Deodorant Ingredients”, S. A. Makin and M. R. Lowry, in “Antiperspirants and Deodorants”, Ed. K. Laden (1999, Marcel Dekker, New York). More preferred anti-microbials for use in the compositions of the invention are polyhexamethylene biguanide salts (also known as polyaminopropyl biguanide salts), an example being Cosmocil CQ™ available from Zeneca PLC, preferably used at up to 1% and more preferably at 0.03% to 0.3% by weight; 2′,4,4′-trichloro,2-hydroxy-diphenyl ether (triclosan), preferably used at up to 1% by weight of the composition and more preferably at 0.05-0.3%; and 3,7,11-trimethyldodeca-2,6,10-trienol (farnesol), preferably used at up to 1% by weight of the composition and more preferably at up to 0.5%.

Other suitable organic antimicrobial agents are transition metal chelators, as described in WO01/52805, for example. Transitional metal chelators having a binding coefficient for iron (III) of greater than 1026, for example diethylenetriaminepentaacetic acid and salts thereof are preferred.

Method and Use

The present invention also provides for a method of reducing perspiration comprising a step of topical application of the composition of the first aspect. Particularly, the present invention provides for a method wherein the composition of the first aspect is applied on the underarms. The present invention also provides for a method wherein topical application of the composition of the first aspect reduces perspiration from the surface of the human body. The method is preferably non-therapeutic. By non-therapeutic is meant that the method is cosmetic in nature.

The invention also provides for use of the composition of the first aspect for reduction of bodily perspiration. The use is preferably non-therapeutic in nature, more preferably cosmetic in nature.

The invention also provides for a composition of the first aspect for for use in providing antiperspirant benefit on a topical surface of the human body.

The invention also provides for use of nanocrystalline cellulose with a length from 1 nm to 300 nm and a diameter from 1 nm to 20 nm wherein surface of said nanocrystalline cellulose comprises hydroxyl and sulfate groups as an antiperspirant agent.

The invention will now be demonstrated with the help of the following non-limiting examples.

### EXAMPLES

Materials

TABLE 1

| Trade/Chemical | | |
|---|---|---|
| name | Particle size | Form |
| Nanocrystalline | Diameter 2.3-4.5 nm, | Powder |
| cellulose | length 44-108 nm | |

### Example 1: Form of Aggregation by Compositions with Model Ionic Sweat

The following compositions were prepared as given in Table-2.

TABLE 2

| Reference | Nanocrystalline | NaCl | | Ionic |
|---|---|---|---|---|
| No. | cellulose (wt %) | (wt %) | Water | strength |
| 1 | 5 | — | Balance to 100 | 0 |
| | | | wt % | |
| 2 | 2 | — | Balance to 100 | 0 |
| | | | wt % | |
| 3 | 2 | 0.0696 | Balance to 100 | 0.012 |
| | | (equal to | wt % | |
| | | 0.012M) | | |
| A | 2 | 0.0928 | Balance to 100 | 0.016 |
| | | (equal to | wt % | |
| | | 0.016M) | | |

The composition of model ionic sweat (pH 6.9) is as given below in Table-3:

TABLE 3

| Ingredient | wt % of total |
|---|---|
| Potassium Chloride | 0.0373 |
| Sodium Bicarbonate | 0.2025 |
| Sodium Chloride | 0.2098 |
| Ammonium Chloride | 0.0107 |
| Calcium Chloride | 0.0222 |
| Lactic Acid | 0.0901 |
| Urea | 0.0018 |
| Water | 99.4256 |

Testing On A Narrow Capillary Apparatus

An experiment was arranged using the device as disclosed in the International Application WO2018099931 A1 (Unilever).

The device has a 20×20 μm channel. Before use, the channel was filled with model ionic sweat. The surface tension of the model ionic sweat is strong and prevents its flowing in the capillary. 20 μl of concerned composition in table-2, was placed at one end of the channel. When the composition contacts with the model ionic sweat, the composition of the present invention is believed to turn into something like an aggregation. This phenomenon simulates the manner in which an antiperspirant active agent forms aggregation thereby provides antiperspirant benefit in the underarm region

The whole process was monitored under optical microscope (Leica™ DM 2500P).

The data on the aggregation formation of the compositions is summarized in Table-4 below:

TABLE 4

| | Aggregation | |
|---|---|---|
| | formation at the end | Time |
| Reference No. | of the channel | (minutes) |
| 1 | Yes | 18 |
| 2 | Yes | 20 |
| 3 | Yes | 20 |
| A | No | 120 |

The data in Table-4 above indicates that compositions as per the invention (Reference No. 1 to 3) can form an aggregation at the end of the 20×20 μm channel when it contacts with the model ionic sweat, which further indicates its ability to form aggregation when used in an antiperspirant composition, while the composition outside of the scope of the present invention (Reference No. A) cannot. In other words, this observation indicates the suitability of the compositions in accordance with the invention to provide antiperspirant benefit like an antiperspirant ingredient.

### Example 2: Effect of Dosage of Nanocrystalline in the Composition on the Performance

The following compositions were prepared as given in Table-5.

TABLE 5

| Reference | Nanocrystalline | | Ionic |
|---|---|---|---|
| No. | cellulose (wt %) | Water | strength |
| B | 0.5 | Balance to 100 wt % | 0 |
| 4 | 1 | Balance to 100 wt % | 0 |
| 5 | 2 | Balance to 100 wt % | 0 |

The composition of model ionic sweat (pH 6.9) is as given in Table-3 above.

Testing On A Narrow Capillary Apparatus

Using the same method as disclosed in Example 1.

The data on the aggregation formation of the compositions is summarized in Table-6 below:

TABLE 6

| | Aggregation | |
|---|---|---|
| | formation at the end | Time |
| Reference No. | of the channel | (minutes) |
| B | No | 120 |
| 4 | Yes | 120 |
| 5 | Yes | 20 |

The data in Table-6 above indicates that compositions as per the invention (Reference No. 4 to 5) can form an aggregation at the end of the 20×20 μm channel when it contacts with the model ionic sweat, which further indicates its ability to form aggregation when used in an antiperspirant composition, while the composition outside of the scope of the present invention (Reference No. B) cannot. In other words, this observation indicates the suitability of the compositions in accordance with the invention to provide antiperspirant benefit like an antiperspirant ingredient.

1. An aqueous antiperspirant composition comprising:
nanocrystalline cellulose with a length from 1 nm to 300 nm and a diameter from 1 nm to 20 nm;
(ii) a cosmetically acceptable carrier,
wherein the ionic strength of the composition is lower than 0.016; wherein the composition is not a pickering emulsion; wherein surface of the nanocrystalline cellulose comprises hydroxyl and sulfate groups, wherein the composition comprises from 2 to 20 wt % of the nanocrystalline cellulose based on total weight of the composition.

2. The aqueous antiperspirant composition as claimed in claim 1, wherein the composition comprises no water-soluble metal salt or a water-soluble metal salt with molarity lower than 0.016.

3. The aqueous antiperspirant composition as claimed in claim 1, wherein surface of the nanocrystalline cellulose is negative charged.

4. The aqueous antiperspirant composition as claimed in claim 1, wherein the nanocrystalline cellulose has a length from 1 nm to 100 nm.

5. The aqueous antiperspirant composition as claimed in claim 1, wherein the composition comprises 2 to 10 wt % of the nanocrystalline cellulose based on total weight of the composition.

6. The aqueous antiperspirant composition as claimed in claim 1, wherein the composition comprises a fragrance.

7. The aqueous antiperspirant composition as claimed in claim 1, wherein the composition comprises an antimicrobial deodorant.

8. The aqueous antiperspirant composition as claimed in claim 1, wherein the composition is in the form of a lotion, spray, firm solid, soft solid, cream, or is an emulsion packaged in a roll-on applicator.

9. The aqueous antiperspirant composition as claimed in claim 1, wherein the composition comprises a propellant and is in the form of an aerosol.

10. A method of reducing perspiration comprising a step of topically applying the aqueous antiperspirant composition as claimed in claim 1.

11. The method as claimed in claim 10, wherein the aqueous antiperspirant composition is applied on the underarms.

12. (canceled)

13. (canceled)

14. The aqueous antiperspirant composition as claimed in claim 2, wherein the water-soluble metal salt is a water-soluble metal salt of sodium, potassium, calcium, magnesium, or zinc.

15. The aqueous antiperspirant composition as claimed in claim 1, wherein the composition comprises 2 to 5 wt % of the nanocrystalline cellulose based on total weight of the composition.

16. The aqueous antiperspirant composition as claimed in claim 1, wherein the composition is free from metal based antiperspirant active.

17. The aqueous antiperspirant composition as claimed in claim 16, wherein the metal based antiperspirant active has a metal that is aluminum, zirconium, zinc, titanium, copper, gallium, stannum, indium, hafnium, vanadium, or cobalt.

18. The aqueous antiperspirant composition as claimed in claim 1, wherein the ionic strength of the composition ranges from 0 to 0.012.

19. The aqueous antiperspirant composition as claimed in claim 1, wherein the ionic strength of the composition is 0.

20. The aqueous antiperspirant composition as claimed in claim 1, wherein the nanocrystalline cellulose has a diameter from 1.5 nm to 10 nm.
